(19) United States
(12) Patent Application Publication
(10) Pub. No.: US 2014/0255907 A1
(43) Pub. Date: Sep. 11, 2014

(54) UNIQUE BUFFERING SYSTEM FOR CELL CULTURE MEDIA AND GAMETE AND EMBRYO CULTURE MEDIA AND METHODS

(71) Applicants: Jeremy G. Thompson, Grange (AU); Aidan McMahon, Newmarket on Fergus (IR); Kim John Giliam, Brisbane (AU)

(72) Inventors: Jeremy G. Thompson, Grange (AU); Aidan McMahon, Newmarket on Fergus (IR); Kim John Giliam, Brisbane (AU)

(21) Appl. No.: 14/196,614

(22) Filed: Mar. 4, 2014

Related U.S. Application Data
(60) Provisional application No. 61/774,363, filed on Mar. 7, 2013.

(51) Int. Cl. C12N 5/073

(52) U.S. Cl. CPC C12N 5/0604
USPC 435/2; 435/404; 435/374

(57) ABSTRACT

The invention relates to compositions and methods for continuous stabilization of pH of cell culture medium notwithstanding change from one distinct environment to another environment. The methods include providing a buffering system that includes a sodium bicarbonate buffer and at least two zwitterionic buffers having concentrations significantly reduced as compared to conventionally used media buffers utilizing a single zwitterionic buffer.

### RELATED APPLICATIONS

The present patent document claims the benefit of the filing date under 35 U.S.C. §119(e) of Provisional U.S. Patent Application Ser. No. 61/774,363, filed Mar. 7, 2013, which is hereby incorporated by reference.

### BACKGROUND

1. Technical Field

The present invention relates to compositions and methods for continuous stabilization of pH of cell culture medium notwithstanding changes in environment conditions.

2. Background Information

Assisted reproduction treatments, such as in vitro fertilization (IVF) seek to duplicate, to a large extent, the conditions and processes normally occurring within the female reproductive system that are necessary for oocyte development, fertilization, and early embryonic development. In the clinic and laboratory, IVF involves several discrete procedures, such as collection of the oocytes from the ovary of the mother, preparation of the sperm, fertilization, and, once fertilized eggs are identified, a period of early embryonic development, and then transfer of the embryo to the mother's uterus. Each of these steps can take place over extended periods of time, during which the individual cells involved have a continuing need for nutrients, and are subjected to significant stress as a result of clinical manipulation and changes environmental conditions.

During IVF, the culture medium is ordinarily used as a substitute for the fluid secreted by the female reproductive tract that would ordinarily surround the gametes, zygote, and developing embryo.

In addition, human and other animal cells are presently being frozen, stored, and then thawed as means of saving these materials for use at a later date. Currently human embryos are frozen and/or vitrified to accomplish cryopreservation. The terms “frozen,” “vitrification” and “cryopreservation” are interchangeable for the purpose of the subject disclosure.

Because the production of healthy preimplantation embryos for assisted reproduction treatments has been a central goal of reproductive technologies, these technologies are continually being refined to aid in this endeavor. The common theme of these diverse modifications in technique is to minimize the stresses imposed upon gametes and embryos within the culture system. Perturbations in environmental variables are often transduced into intracellular instabilities that lead to devastating effects on embryo quality and viability. One such stressor, easily introduced in the laboratory, is fluctuation of extracellular pH.

A multitude of intracellular processes are dependent upon pH, and therefore, the maintenance of stable pH is important for optimizing cell culture. For example, pH is intimately involved with regulation of embryo cytoskeletal dynamics. Cytoskeletal elements are not only responsible for cytokines, but also instrumental in positioning organelles, such as mitochondria. Proper mitochondrial polarization is especially important with regard to oocyte developmental competence. pH also directly influences cellular metabolism (Busa and Nuccitelli (1984)). Even small increases in pHᵢ perturbs embryo metabolism (Edwards et al., (1998); Lane et al., (2000)), which can profoundly affect subsequent development. Thus, as is the case for embryos (Leclerc et al., (1994); Zhao et al., (1995); Zhao and Baltz, (1996); Lane and Bavister, (1999); Lane et al. (1999a)), instability in pH conditions used for oocyte manipulations may have detrimental consequences (Bagger et al. (1987).

Some of the current handling media utilize single buffers, such as MOPS or HEPES. However, the use of single buffer limits the ability to adjust the range of buffering capacity. Furthermore, changes in temperature alter buffering of these compounds. Therefore, traditional IVF handling media utilizing a single buffer may not provide ideal pH buffering. As such, stability of pH of the culture media during cell culture handling procedures, such as IVF procedures is considered an essential concept when fertilizing and culturing embryos in vitro (Swain and Pool, RBMOnline, 2009).

Swain and Pool suggested that combining multiple buffers, such as HEPES and MOPS and/or DIPSO, into a single medium in various ratios gives the ability to shift the effective buffering range to cover a specific pH compared with media containing only a single buffer (Swain and Pool, RBMOnline, 18(6):799-810 (2009)). Although, Swain and Pool suggested that this would also allow for simultaneous reduction of absolute concentrations of these individual buffers to alleviate possible toxicity, the authors do not provide any specific recommendations for concentrations of the buffers that would be suitable for use with human cell lines. Also, specific, preferable combinations and concentrations of the zwitterionic buffers in the preferable combinations are not disclosed. Moreover, the given concentrations and mixing of buffers was only studied in connection with non-culture embryo handling scenarios. The effect of the multitude of zwitterionic buffers in combination at distinct concentrations in combination with sodium bicarbonate buffer was also not studied or suggested.

U.S. Pat. No. 5,747,341 to Brothers suggested use on HEPES and MOPS in a formula for long term culture of pancreatic islet cells. However, the suggested concentrations of the buffers were high and not suitable for the oocyte cultures or other in vitro applications.

As such, there is still a need for improved cell buffering methods and systems.

### SUMMARY

One embodiment relates to a method for continuous stabilization of pH of cell culture medium notwithstanding change from the first environment conditions to the second environment conditions. The method includes providing a buffering system, the buffering system comprising sodium bicarbonate at a concentration from 4 to 30 mM, and at least two zwitterionic buffers at a concentration from 1 to 50 mM, where the pH is 7.0-7.6, and where the first and the second environment conditions are distinct. In the method, the zwitterionic buffers may be 3-N-morpholinopropane sulfonic acid (MOPS); N-2-Hydroxyethylpiperazine-N′-2-ethanesulfonic acid (HEPES); and N,N-bis2-Hydroxyethyl-2-aminoethanesulfonic acid (BES); 1,3-bistris(Hydroxymethyl)methylaminopropane (BIS-TRIS PROPANE); 2-bis(2-Hydroxyethyl)aminoethanesulfonic acid); (N-trisHydroxymethylmethyl-2-aminoethane-sulfonic acid (TES); 2-(2-Hydroxy-1,1-bis(hydroxymethyl)ethylamino)ethanesulfonic acid; 3-N,N-bis(2-Hydroxyethyl)methylamino-2-hydroxy-propanesulfonic acid) (DIPSO); 3-N-tris(Hydroxymethyl)methylamino-2-hydroxypropanesulfonic acid) (TAPSO); trisHydroxymethylaminomethane),(2-amino-2-(hydroxymethyl)-1,3-propanediol) (TRIZMA); N-2-Hydroxyethylpiperazine-N′-2-hydroxy-propanesulfonic acid (HEPPSO); piperazine-N,N′-bis2-hydroxypropanesulfonic acid (POPSO); N2-Hydroxyethylpiperazine-N′-3-propane-sulfonic acid (HEPPS); and Triethanolamine, (2,2′2″-Nitrilotriethanol) (TEA). In the method, the first environment conditions may be CO₂-enriched environment conditions and the second environment conditions may be ambient environment conditions; the first environment conditions may be CO₂-enriched environment conditions and the second environment conditions may be CO₂-reduced environment conditions; the first environment conditions may be ambient environment conditions and the second environment conditions may be CO₂-enriched environment conditions; the first environment conditions may be ambient environment conditions and the second environment conditions may be CO₂-reduced environment conditions; the first environment conditions may be CO₂-reduced environment conditions and the second environment conditions may be ambient environment conditions; the first environment conditions may be CO₂-reduced environment conditions and the second environment conditions may be CO₂-enriched environment conditions.

Another embodiment provides a cell culture buffering system for continuous stabilization of pH of cell culture medium from first environment conditions to second environment conditions. The buffering system includes 4 to 30 mM of sodium bicarbonate, and at least two zwitterionic buffers at a concentration from 1 to 50 mM, where the pH is 7.0-7.6, and where the first and the second environment conditions are distinct. Also, the cell culture buffering system may include 7.5 nM 3-N-morpholinopropane sulfonic acid (MOPS), 7.5 nM 3-N-morpholinopropane sulfonic acid (MOPS), and 25 nM NaHCO₃. In the cell culture buffering system the zwitterionic buffers may be selected from 3-N-morpholinopropane sulfonic acid (MOPS); N-2-Hydroxyethylpiperazine-N′-2-ethanesulfonic acid (HEPES); and N,N-bis2-Hydroxyethyl-2-aminoethanesulfonic acid (BES); 1,3-bistris(Hydroxymethyl)methylaminopropane (BIS-TRIS PROPANE); 2-bis(2-Hydroxyethyl)aminoethanesulfonic acid); (N-trisHydroxymethylmethyl-2-aminoethane-sulfonic acid (TES); 2-(2-Hydroxy-1,1-bis(hydroxymethyl)ethylamino)ethanesulfonic acid; 3-N,N-bis(2-Hydroxyethyl)methylamino-2-hydroxy-propanesulfonic acid) (DIPSO); 3-N-tris(Hydroxymethyl)methylamino-2-hydroxypropanesulfonic acid) (TAPSO); trisHydroxymethylaminomethane),(2-amino-2-(hydroxymethyl)-1,3-propanediol) (TRIZMA); N-2-Hydroxyethylpiperazine-N′-2-hydroxy-propanesulfonic acid (HEPPSO); piperazine-N,N′-bis2-hydroxypropanesulfonic acid (POPSO); N2-Hydroxyethylpiperazine-N′-3-propane-sulfonic acid (HEPPS); and Triethanolamine, (2,2′2″-Nitrilotriethanol) (TEA). The buffering system may be suitable for use to support the development of cells. The buffering system may be suitable for use to support the development of embryos. The buffering system may be suitable for use to support the development of zygotes. The buffering system may be for use as a cryopreservation buffer or an in-vitro maturation buffer. The buffering system may be for use with follicular cultures. The buffering system may be for use in assisted reproduction procedures. The buffering system may be for use with granulosa cells, theca cells, cumulus cells, germinal vesicle oocytes, methaphase I oocytes, and metaphase II oocytes and all stages of preimplantation embryo development and stem cells derived from any of embryonic cells or cells either collected from tissue or induced from any non-stem cell.

Yet, further embodiment relates to a method for continuous stabilization of pH of cell culture medium comprising ejaculated sperm cells, notwithstanding change from the first environment conditions to the second environment conditions. The method includes providing a buffering system, where the buffering system comprising sodium bicarbonate at a concentration from 4 to 30 mM, and at least two zwitterionic buffers at a concentration from 1 to 50 mM, wherein the pH is 7.0-7.6, and wherein the first and the second environment conditions are distinct. In the method, the sperm cells are in a non-cryopreserved state. In the method, the buffering system provides for a continuous stabilization of pH of the cell culture medium over a time period of about 24 to about 48 hours without a temperature control.

Yet, another embodiment relates to a method for transporting ejaculated sperm cells in a cell culture medium from a first environment to a second environment without a temperature control and notwithstanding change from the first environment conditions to the second environment conditions. The method includes providing a buffering system, the buffering system comprising sodium bicarbonate at a concentration from 4 to 30 mM, and at least two zwitterionic buffers at a concentration from 1 to 50 mM, wherein the cell medium has a pH of 7.0-7.6, and wherein the first and the second environment conditions are distinct.

### DESCRIPTION OF THE DRAWINGS

FIG. 1A-B depicts effect on pH when 6% CO₂ equilibrated media is allowed to degas overtime.

FIG. 2A-B depicts a graph showing the effect on pH when 6% CO₂ media is allowed to degas overtime at 37° C. (A) or at room temperature (B).

### DETAILED DESCRIPTION OF THE DRAWINGS AND THE PRESENTLY PREFERRED EMBODIMENTS

It has been recognized by the applicants that, cell culturing, including IVF processes, as well the cryopreservation may be improved by providing continuous stabilization of pH of the cell culture medium whether the medium is in a CO₂-enriched environment (e.g., an incubator), an unconstrained or uncontrolled environment (e.g., ambient environment outside of the incubator), or a CO₂-reduced environment (e.g., during cryopreservation procedures or freezing). Such stabilization can be achieved by providing the buffering system described herein.

Without being bound by a specific mechanism, it is believed that having at least two zwitterionic buffers present in the cell culture media at significantly lower concentrations as compared to conventional buffers, in addition to a sodium bicarbonate component allows for maintenance of stable pH for optimizing cell culture. Applicants surprisingly found that pH of the buffered medium, according to the methods described herein, had significant stability during cell culturing in a CO₂-enriched environment, in an unconstrained or uncontrolled environment (e.g., during a procedure that requires removal of the cell culture from an incubator), or a CO₂-reduced environment (e.g., during and following cryopreservation procedure).

Various terms that will be used throughout the specification have meanings that will be well understood by a skilled addressee. However, for ease of reference, some of these terms will now be defined.

It should be understood that the terms “a” and “an” as used above and elsewhere herein refer to “one or more” of the enumerated components. For example, “a” cell refers to one cell or a mixture comprising two or more cells.

As used herein the terms “comprise(s),” “include(s),” “having,” “has,” “contain(s),” and variants thereof, are intended to be open-ended transitional phrases, terms, or words that do not preclude the possibility of additional acts or structure.

The term “pH” refers to a measure of the acidity or alkalinity of a solution equal to the common logarithm of the reciprocal of the concentration of hydrogen ions in moles per cubic decimeter of solution. For example, pure water has pH of 7 (neutral), acid solutions have pH less than 7, and alkaline solutions have pH greater than 7. The pH scale commonly in use ranges from 0 to 14.

The terms “buffer,” “buffering system,” and/or “buffer solution” refer to a solution, which reduces the change of pH upon addition of small amounts of acid or base, or upon dilution. The term “buffering agent” refers to a weak acid or weak base in a buffer solution.

The term “reduced concentration” refers to a concentration of a reagent or a component of the culture media buffering system that is lower than a concentration of the same reagent in a conventionally used buffer. For example, the concentration of both HEPES or MOPS buffers are conventionally in the order of 20-25 mM when oocytes, embryos or cells are maintained in air without supplemental CO₂. Concentrations lower than this would be a “reduced concentration.”

The term “continuous” in connection with the pH means that the pH of a solution or a buffer, such as the buffering system described herein remains the same (i.e., identical in value) or close to the same (i.e., close to identical or +/−0.3 pH units), in at least two varying environmental conditions, such as the CO₂-enriched environment and ambient environment. For example, if the pH of the cell medium in an incubator (i.e., CO₂-enriched environment) is 7.3, it will remain at within +/−0.3 pH units during manipulation of the cell culture outside of the incubator (i.e., ambient environment) following the incubation in the CO₂-enriched environment.

The terms “stable” and “stabilization” mean resistant to change of condition; not easily disturbed or subject to sudden or extreme change or fluctuation; self-restoring. Specifically, in the context of this application, “stable pH” of a cell culture medium means that the pH is resistant to change in the environment and will remain the same or similar throughout the change in the environments (e.g., it will remain the same or similar in the incubator as well as when placed outside of the incubator).

The term “modulating” means any inhibition or augmentation of a process, or any inhibition or augmentation of the activity, function or characteristic of a particular entity.

The term “CO₂-enriched environment” refers to a place or an environment containing higher concentration of CO₂ as compared to ambient or atmospheric environment. For example, a cell culture incubator may be one example of an enriched CO₂ environment, the incubator having, e.g., 6% carbon dioxide.

The term “ambient environment” refers to an environment with atmospheric conditions (i.e., dry air contains roughly (by volume) 78.09% nitrogen, 20.95% oxygen, 0.93% argon, 0.039% carbon dioxide, and small amounts of other gases in addition to a variable amount of water vapor, on average around 1%). For example, a room where cell culture manipulations occur may be one example of an ambient environment.

The term “CO₂-reduced environment” refers to a place or an environment containing lower concentration of CO₂ as compared to ambient or atmospheric environment (i.e., lower than 0.039% carbon dioxide). For example, a cryopreservation freezer may be one example of a reduced CO₂ environment.

The term “cells” refers to somatic and germline cells.

The term “somatic cell” refers to any cell forming the body of an organism, as opposed to germline cells. Somatic cells may be of mammalian and non-mammalian origin. For example, in mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells.

The term “oocyte” refers to a cell from which an egg or ovum develops by meiosis; a female gametocyte.

The terms “fertilized oocyte”, “zygote” or “zygocyte” refer to the initial cell synthesized from the union of two gametes, and constitutes the first stage in a unique organism's development. In multicellular organisms, it is the earliest developmental stage of the embryo. Zygotes are usually produced by a fertilization event between two haploid cells—an ovum (female gamete) and a sperm cell (male gamete)—which combine to form the single diploid cell. Such zygotes contain DNA derived from both the parents, and this provides all the genetic information necessary to form a new individual.

The term “embryo” refers to a multicellular diploid eukaryote in its earliest stage of development, from the time of first cell division until birth. In humans, it is called an embryo until about eight weeks after fertilization, and from then it is instead called a fetus.

The terms “embryonic cell,” “embryonic stem cell,” or “pluripotent stem cell,” may be used interchangeably and refer to one of the cells that are self-replicating, are derived from embryos, such as human embryos or human fetal tissue, and are known to develop into cells and tissues of the three primary germ layers. Although human pluripotent stem cells may be derived from embryos or fetal tissue, such stem cells are not themselves embryos. (See the National Institutes of Health Guidelines for Research Using Human Pluripotent Stem Cells.) “Self-replicating” means the cell can divide and form cells indistinguishable from it. The “three primary germ layers”—called the ectoderm, mesoderm, and endoderm—are the primary layers of cells in the embryo from which all tissues and organs develop.

The term “embryo development” refers to the cleavage of a newly formed zygote through several cell cycle divisions which during the course of these divisions, the embryo initiates significant levels of gene expression that results in the process of cellular differentiation being initiated. This initial differentiation is the formation of an outer trophectoderm cell layer and an inner cell mass, at which stage the embryo is described as a “blastocyst.” Development continues to form the ectoderm, mesoderm and endoderm.

The term “follicle development” and variants thereof as used throughout the specification is to be understood to mean the progression of an ovarian follicle through the stages of a primordial follicle to a preovulatory follicle through to the corpus luteum. In this regard, it will be understood that the follicle may be present in an entire female subject, or alternatively may be present in vitro, such as a follicle isolated from a female subject.

The term “oocyte maturation” and variants thereof as used throughout the specification is to be understood to mean the process whereby an oocyte progresses from a meiotically immature state, being incapable of being fertilized, to an oocyte that is meiotically mature, being fertilizable and capable of producing a viable embryo. The term will be understood to also include maturation of oocyte cytoplasm, such that the oocyte is able to support embryo development post-fertilization. In this regard, it will be understood that the oocyte may be present in an entire female subject, or alternatively may be present in vitro, such as an oocyte isolated from a female subject.

The term “assisted reproduction” as used throughout the specification is to be understood to mean any fertilization technique in humans and animals involving isolated oocytes and/or isolated sperm, including a technique using an oocyte or embryo cultured in vitro (for example in vitro maturation of an oocyte), in vitro fertilization (IVF; aspiration of an oocyte, fertilization in the laboratory and transfer of the embryo into a recipient), gamete intrafallopian transfer (GIFT; placement of oocytes and sperm into the fallopian tube), zygote intrafallopian transfer (ZIFT; placement of fertilized oocytes into the fallopian tube), tubal embryo transfer (TET; the placement of cleaving embryos into the fallopian tube), peritoneal oocyte and sperm transfer (POST; the placement of oocytes and sperm into the pelvic cavity), intracytoplasmic sperm injection (ICSI), testicular sperm extraction (TESE), and microsurgical epididymal sperm aspiration (MESA).

The term “isolated” as used in relation to oocytes and embryos is to be understood to mean that the oocyte or embryo has at some time been removed or purified (at least partially) from its natural environment. An example of an isolated embryo is an embryo produced in vitro using an assisted reproduction technology or an embryo isolated from a subject. An example of an isolated oocyte is an oocyte isolated from a subject as part of a follicle, a cumulus oocyte complex, or a denuded oocyte.

The term “developmentally competent” is to be understood to mean an embryo or oocyte that is capable of forming an embryo that is capable of implantation.

The term “developmental competence” is to be understood to mean the ability of an oocyte or embryo to develop into an embryo capable of implantation. An oocyte or embryo with improved developmental competence will have an increased probability that it will develop into a live animal or human after successful implantation.

One embodiment relates to a method for continuous stabilization of pH of cell culture medium, notwithstanding change from the first environment conditions to the second environment conditions that includes providing a cell culture medium buffering system that includes a sodium bicarbonate buffer and a combination of at least two zwitterionic buffers.

Preferably, the sodium bicarbonate buffer has a sodium bicarbonate at a concentration in the range from 10 mM to 60 mM. More preferably, the concentration of sodium bicarbonate is 20 mM to 40 mM. Most preferably, the concentration of sodium bicarbonate is 25 mM.

At least two zwitterionic buffers are included in the buffering system of this invention. Preferably, the zwitterionic buffer is chosen from the group including 3-N-morpholinopropane sulfonic acid (called “MOPS”); N-2-Hydroxyethylpiperazine-N′-2-ethanesulfonic acid (called “HEPES”); and N,N-bis2-Hydroxyethyl-2-aminoethanesulfonic acid (called “BES”).

Although these three zwitterionic buffers mentioned directly above are the preferred buffers, being most suitable over the pH range required for “embryo comfort,” other zwitterionic buffers may be used, and it is noted that the following zwitterionic buffers can also be adapted for use in the preferred pH range of about 7.2-7.4:
BIS-TRIS PROPANE (1,3-bistris(Hydroxymethyl)methylaminopropane);
BES (N,N-bis2-Hydroxyethyl-2-aminoethanesulfonic acid);
2-bis(2-Hydroxyethyl)aminoethanesulfonic acid);
TES (N-trisHydroxymethylmethyl-2-aminoethane-sulfonic acid;
2-(2-Hydroxy-1,1-bis(hydroxymethyl)ethylamino)ethanesulfonic acid;
DIPSO 3-N,N-bis(2-Hydroxyethyl)methylamino-2-hydroxy-propanesulfonic acid);
TAPSO (3-N-tris(Hydroxymethyl)methylamino-2-hydroxypropanesulfonic acid);
TRIZMA (trisHydroxymethylaminomethane),(2-amino-2-(hydroxymethyl)-1,3-propanediol);
HEPPSO(N-2-Hydroxyethylpiperazine-N′-2-hydroxy-propanesulfonic acid);
POPSO (piperazine-N,N′-bis2-hydroxypropanesulfonic acid);
EPPS(N2-Hydroxyethylpiperazine-N′-3-propane-sulfonic acid; HEPPS);
TEA Triethanolamine, (2,2′2″-Nitrilotriethanol).

Because multiple forms of zwitterionic buffers are used in combination with sodium bicarbonate, the zwitterionic buffers are suitably used at reduced concentrations, as compared to conventional buffering system that include only one zwitterionic buffer. Preferred concentration range of the zwitterionic buffers in the buffering system is 1-50 mM; more preferably 1-25 mM. Preferably, the total concentration of the zwitterionic buffer will not exceed 50 mM. For example, if 50 zwitterionic buffers are present in the buffering system described herein, each will be at 1 mM; if 4 zwitterionic buffers are present in the buffering system, each will be at 12.5 mM that do not exceed 50 mM in total.

The exemplified concentration ranges for the zwitterionic buffers are significantly lower in the buffering system described herein as compared to the concentrations of the same zwitterionic buffers in conventional buffers.

In one preferable embodiment, the buffering system may include a sodium bicarbonate buffer and at least two zwitterionic buffers, such as HEPES and MOPS. Preferably, the sodium bicarbonate buffer has a sodium bicarbonate concentration of 25 mM. Preferably, the HEPES is at a concentration of 7.5 mM and MOPS is at a concentration of 7.5 mM. The buffering system is for a continuous stabilization of pH of cell culture medium notwithstanding change from the first environment conditions to the second environment conditions, where the first and second environment conditions are distinct, where pH never exceeds 7.55.

The buffering system may further include additional components. For example, a carbon source may be included in the buffering system described herein. Preferably the carbon source is chosen from the class of sugars, and more preferably it is glucose. Preferably the glucose is in the range of 0.01-1.0 g/L.

In certain embodiments, where the buffering system is used as an embryo holding solution, it is preferred that the solution also contains an effective amount of albumin. However in the case of an embryo flushing solution based on these buffers then the albumin is omitted.

In certain embodiments, the buffering system may contain sodium chloride in the range of 1.0-10.0 g/L.

In certain embodiments, the buffering system may contain potassium chloride in the range of 0.1-1.0 g/L.

Preferably, the water using in the buffering system is tripled distil led water and is of purity sufficient for embryo holding solutions, typically referred to as “tissue culture grade water.”

In certain additional embodiments, the buffering system further includes pyruvate, lactate and/or amino acids.

Preferably, the concentration of NaCl in the composition is 100 mM to 180 mM. Most preferably, the concentration of NaCl is 140 mM.

Preferably, the concentration of KCl in the composition is 1 mM to 8 mM. Most preferably, the concentration of KCl is 4 mM.

Preferably, the concentration of glucose in the composition is 1 mM to 25 mM. Most preferably, the concentration of glucose is less than or equal to 5.6 mM.

For example, an exemplary cell medium formulation of the buffering system described herein may include the following reagents:

TABLE 2

| Reagents | mM |
|---|---|
| Sodium Chloride (NaCl) | 92.5 |
| Potassium Chloride (KCl) | 6 |
| Magnesium Sulphate (MgSO₄) | 0.5 |
| Sodium Phosphate Monobasic (NaH₂PO₄) | 0.4 |
| Calcium L-lactate pentahydrate (C₆H₁₀CaO₆) | 1.5 |
| NaPyruvate | 0.3 |
| Glucose | 2 |
| Fructose | 0.5 |
| Taurine (C₂H₇NO₃S) | 1 |
| Glycine | 2 |
| Analyl-glutamine | 1 |
| Carnitine | 1 |
| Asparagine•H₂O | 0.05 |
| Aspartate | 0.1 |
| Glutamate | 0.15 |
| Proline | 0.1 |
| Serine | 0.1 |
| Arginine•HCl | 0.1 |
| Cystine | 0.1 |
| Histidine•HCl | 0.05 |
| Iso-Leucine | 0.1 |
| Leucine | 0.1 |
| Lysine•HCl | 0.1 |
| Methionine | 0.05 |
| Phenylalanine | 0.1 |
| Threonine | 0.05 |
| Tryptophan | 0.05 |
| Valine | 0.1 |
| Tyrosine | 0.05 |
| EDTA | |
| MOPS | 7.5 |
| HEPES | 7.5 |
| NaHCO₃ | 25 |
| HSA (mg/ml) | 4 |
| Gentamicin (mg/ml) | 0.1 |

In another embodiment, an exemplary cell medium formulation of the buffering system may include the following reagents:

TABLE 3

| Reagents | mM |
|---|---|
| Sodium Chloride (NaCl) | 92.5 |
| Potassium Chloride (KCl) | 6 |
| Magnesium Sulphate (MgSO₄) | 0.5 |
| Sodium Phosphate Monobasic (NaH₂PO₄) | 0.4 |
| Calcium L-lactate pentahydrate (C₆H₁₀CaO₆) | 1.5 |
| NaPyruvate | 0.3 |
| Glucose | 2 |
| Fructose | 0.5 |
| Glycine | 2 |
| Analyl-glutamine | 1 |
| Asparagine•H₂O | 0.05 |
| Aspartate | 0.1 |
| Glutamate | 0.15 |
| Proline | 0.1 |
| Serine | 0.1 |
| Arginine•HCl | 0.1 |
| Cystine | 0.1 |
| Histidine•HCl | 0.05 |
| Iso-Leucine | 0.1 |
| Leucine | 0.1 |
| Lysine•HCl | 0.1 |
| Methionine | 0.05 |
| Phenylalanine | 0.1 |
| Threonine | 0.05 |
| Tryptophan | 0.05 |
| Valine | 0.1 |
| Tyrosine | 0.05 |
| EDTA | |
| MOPS | 7.5 |
| HEPES | 7.5 |
| NaHCO₃ | 25 |
| HSA (mg/ml) | 4 |
| Gentamicin (mg/ml) | 0.1 |

The buffering system allows for the pH to stay in range from 7.0 to 7.6; preferably in the range from 7.1 to 7.6; and most preferably in the range from 7.2 to 7.6.

In one embodiment, the invention is directed to a method for continuous stabilization of pH of cell culture medium notwithstanding change from first environment conditions to second environment conditions, comprising providing a buffering system, the buffering system comprising a sodium bicarbonate buffer and at least two zwitterionic buffers, wherein the pH is remains in range 7.0-7.6, where the first and the second environment conditions are distinct.

For example, in certain embodiments, the first environment conditions are CO₂-enriched environment conditions and the second environment is ambient environment conditions.

In another embodiment, the first environment conditions are CO₂-enriched environment conditions and the second environment CO₂-reduced environment conditions.

In another embodiment, the first environment conditions are ambient environment conditions and the second environment conditions are CO₂-enriched environment conditions.

In another embodiment, the first environment conditions are ambient environment conditions and the second environment conditions are CO₂-reduced environment conditions.

In another embodiment, the first environment conditions are CO₂-reduced environment conditions and the second environment conditions are ambient environment conditions.

In yet another embodiment, the first environment conditions are CO₂-reduced environment conditions and the second environment conditions are CO₂-enriched environment conditions.

In one embodiment, the invention is directed to a method for continuous stabilization of pH of cell culture medium notwithstanding change from first environment conditions to second environment conditions to third environment conditions. For example, the first environment conditions may be CO₂-enriched environment conditions, the second environment conditions may be ambient environment conditions, and the third environment conditions may be CO₂-reduced environment conditions.

In another embodiment, the cell culture buffering system for continuous stabilization of pH of cell culture medium from first environment conditions to second environment conditions include providing a buffering system comprising from 2 to 30 mM of bicarbonate; from 1 to 50 mM of HEPES; and from 1 to 50 mM of MOPS.

Virtually any suitable culture medium to cultivate, grow, recover, multiply, isolate, cryopreserve, develop and/or progress cellular culture may be used in conjunction with the buffering system described herein. Some exemplary cell culture buffers include Tissue Culture Medium 199, Alpha Minimal Essential Medium, Hams F10 and F12, and Waymouth's medium.

In one embodiment of the invention, the buffering system is for use with cells, such as oocytes, granulosa cells, theca cells, cumulus cells, germinal vesicles, methaphase I, and metaphase II cells, embryos and zygotes, but not limited to these cells. Specifically, the buffering system may be used for culturing, development and maturation of cells, such as oocytes, granulosa cells, theca cells, cumulus cells, germinal vesicles, methaphase I, and metaphase II cells, embryos and zygotes.

In certain embodiments, the cell culture medium buffering system may be suitable for use with a cumulus oocyte complex and/or a follicle. Accordingly, one embodiment relates to a cell culture medium buffering system for culturing of a cumulus oocyte complex and/or a follicle, the cell culture medium buffering system including one or more of the following:

(i) bicarbonate at a concentration from 4 to 30 mM, and
(ii) at least two zwitterionic buffers at a concentration from 1 to 50 mM.

In certain embodiments, the cell culture medium buffering system may be suitable for use with zygote. Accordingly, one embodiment relates to a cell culture medium buffering system for culturing of a zygote, the cell culture buffering system including one or more of the following:

(i) bicarbonate at a concentration from 4 to 30 mM, and
(ii) at least two zwitterionic buffers at a concentration from 1 to 50 mM.

In certain embodiments, the cell culture buffering system may be suitable for use with an embryo. Accordingly, one embodiment provides a cell culture buffering system for culturing of an embryo, the cell culture buffering system including one or more of the following:

(i) bicarbonate at a concentration from 4 to 30 mM, and
(ii) at least two zwitterionic buffers at a concentration from 1 to 50 mM.

In certain embodiments, the cell culture buffering system may be suitable for use with a cumulus oocyte complex and/or a follicle. Accordingly, one embodiment provides a cell culture buffering system for development of a cumulus oocyte complex and/or a follicle, the cell culture buffering system including one or more of the following:

(i) bicarbonate at a concentration from 4 to 30 mM, and
(ii) at least two zwitterionic buffers at a concentration from 1 to 50 mM.

In certain embodiments, the cell culture buffering system may be suitable for use with a zygote. Accordingly, one embodiment provides a cell culture buffering system for development of a zygote, the cell culture buffering system including one or more of the following:

(i) bicarbonate at a concentration from 4 to 30 mM, and
(ii) at least two zwitterionic buffers at a concentration from 1 to 50 mM.

In certain embodiments, the cell culture buffering system may be suitable for use with an embryo. Accordingly, one embodiment provides a cell culture buffering system for development of an embryo, the cell culture buffering system including one or more of the following:

(i) bicarbonate at a concentration from 4 to 30 mM, and
(ii) at least two zwitterionic buffers at a concentration from 1 to 50 mM.

In another embodiment, the cell culture buffering system may be used for the in vitro maturation of an oocyte in a cumulus oocyte complex. Accordingly, another embodiment provides an oocyte in vitro maturation cell culture buffering system, the cell culture buffering system including one or more of the following components:

(i) bicarbonate at a concentration from 4 to 30 mM, and
(ii) at least two zwitterionic buffers at a concentration from 1 to 50 mM.

In another embodiment, the cell culture buffering system may be used for the in vitro maturation of a zygote. Accordingly, another embodiment provides a zygote in vitro maturation cell culture buffering system, the cell culture buffering system including one or more of the following components:

(i) bicarbonate at a concentration from 4 to 30 mM, and
(ii) at least two zwitterionic buffers at a concentration from 1 to 50 mM.

In another embodiment, the cell culture buffering system may be used for the in vitro maturation of an embryo. Accordingly, another embodiment provides an embryo in vitro maturation cell culture buffering system, the cell culture buffering system including one or more of the following components:

(i) bicarbonate at a concentration from 4 to 30 mM, and
(ii) at least two zwitterionic buffers at a concentration from 1 to 50 mM.

The cell culture buffering system is also suitable as a medium for culturing a follicle, and in particular, to improve follicle development or reduce follicle atresia. Accordingly, another embodiment provides a cell culture buffering system as a follicle culture medium.

Methods for the use of such compositions or media for these purposes are known on the art.

In an additional embodiment, the cell buffering system described herein may be used as holding solution and/or a flushing solution. The term “holding solution” refers to a solution used for a short term incubation prior to a longer culture/maturation step; the term “flushing solution” refers to a solution used to wash the contents of a follicle during oocyte collection.

In another embodiment, the buffering system may also be used during the cryopreservation procedures. Specifically, the use of the buffering system may allow for reducing apoptosis induced by freezing and freeze-thawing damage.

Accordingly, another embodiment relates to a cell buffering composition for reducing granulosa cell apoptosis due to cryopreservation and freeze-thawing, the composition including bicarbonate component and at least two zwitterionic buffers.

The composition may be used before freezing or cryopreservation and/or after thawing. For example, individual cumulus oocyte complexes, whole follicles, ovarian tissue, or whole ovaries when frozen typically die as a result of freeze/thawing, and the composition may be used to improve the viability of cells and tissue following freeze-thawing or cryopreservation.

Accordingly, another embodiment provides a buffering system for reducing damage to a cumulus oocyte complex, follicle, ovarian tissue or ovary due to freezing, the composition including a sodium bicarbonate buffer component and at least two zwitterionic buffers. The concentrations of sodium bicarbonate and the zwitterionic buffers may be as outlined above.

In a preferred from, the buffering system is a culture medium.

The buffering system and/or medium is particularly suitable for culturing oocytes that are used for assisted reproduction technologies. Methods for performing assisted reproduction are known in the art.

Accordingly, another embodiment provides a method of assisted reproduction involving an oocyte, the method including the step of culturing the oocyte in a buffering system comprising a bicarbonate component and at least two zwitterionic buffers.

For example, the buffering system described herein may be used during or in an in vitro fertilization technique. Accordingly, in another embodiment, the buffering system may be used for in vitro fertilization of an oocyte.

Further embodiment also provides a buffering system for use in assisted reproduction involving an oocyte.

Yet another embodiment provides a method of assisted reproduction involving an embryo produced from an oocyte, the method including the step of culturing the oocyte and/or the embryo in a buffering system described herein including a bicarbonate component and at least two zwitterionic buffers.

Another embodiment provides a buffering system for use in assisted reproduction involving an embryo produced from an oocyte. In a preferred form, there is also provided a buffering system for improving developmental competence of an oocyte, the buffering system comprising a bicarbonate component and at least two zwitterionic buffers.

Yet another embodiment provides for a buffering system that may be used with a media used to transport ejaculated sperm cells without any temperature control over a period of about 24 hours to about 48 hours; alternatively, about 24 hours to about 72 hours or longer. The use of the buffering system described herein allows for transporting the ejaculated sperm cells in a non-cryopreserved state.

Certain embodiments relate to a method for continuous stabilization of pH of cell culture medium that includes ejaculated sperm cells, notwithstanding change from the first environment conditions to the second environment conditions. The method includes providing a buffering system, the buffering system comprising sodium bicarbonate at a concentration from 4 to 30 mM, and at least two zwitterionic buffers at a concentration from 1 to 50 mM, wherein the pH is 7.0-7.6, and wherein the first and the second environment conditions are distinct.

Certain other embodiments relate to a method for transporting ejaculated sperm cells in a cell medium from a first environment to a second environment without a temperature control and notwithstanding change from the first environment conditions to the second environment conditions. The method includes providing a buffering system, the buffering system comprising sodium bicarbonate at a concentration from 4 to 30 mM, and at least two zwitterionic buffers at a concentration from 1 to 50 mM, wherein the cell medium has a pH in a range 7.0-7.6 and remains in the range during the transport from the first environment to the second environment, and wherein the first and the second environment conditions are distinct.

### EXAMPLES

For Examples 1 and 2, the following HEPES/MOPS buffering medium was used:

| | mM | MW | mg/100 ml |
|---|---|---|---|
| MOPS Na salt | 4 | 231.2 | 92 |
| MOPS acid | 3.5 | 209.3 | 73 |
| HEPES Na salt | 3.75 | 260.29 | 98 |
| HEPES acid | 3.75 | 238.3 | 89 |
| NaHCO3 | 25 | 84.01 | 210 |

### Example 1

### Mixed Buffer Approach

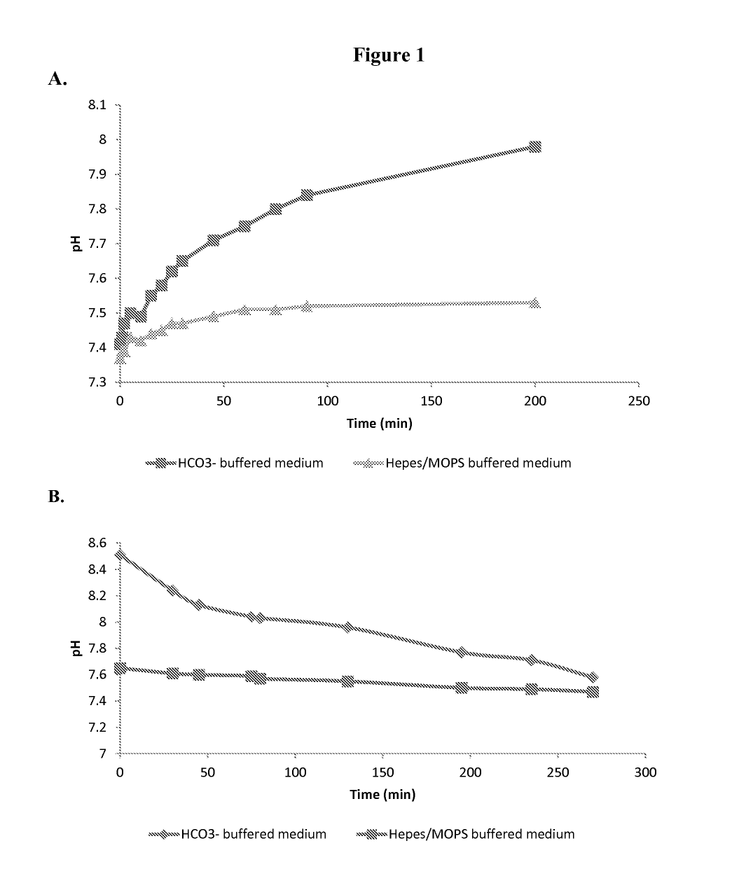

Stability of pH of the buffering system (i.e., HEPES/MOPS buffering medium) as compared to the conventional media (i.e., HCO₃⁻ buffered medium) was measured. Preliminary results using the mixed buffer approach (of the present invention) for the stability of pH when media is removed from a 6% CO₂ incubator are illustrated in FIG. 1A-B.

When equilibrated overnight and then allowed to degas in air, over a 3 hour period, the mixed buffering system never exceeded pH 7.55. In contrast, over the same period, the HCO₃⁻ system behaved as expected, and reached pH 8 (FIG. 1A).

Furthermore, when the pH of the mixed buffer medium was determined after >24 hour period of un-gassed storage, the pH remained below pH 7.7 (FIG. 1B). This suggests that stability of a critical parameter, pH of culture media may be controlled with use of the mixed buffer medium of the present invention during embryo development.

### Example 2

### Study of the BSA-Enriched Buffering System

To assess whether the new buffering system was suitable to support development of cell zygotes, mouse CBA×C57Bl6 1-cell embryos were incubated for 24 hours in a the HEPES/MOPS buffering medium (described above), which was BSA-enriched, amino acid free. The mouse CBA×C57Bl6 1-cell embryos were then moved to a BSA-free medium. This medium was highly suboptimal for development of cell zygotes. A conventional control medium was also used.

As shown in Table 4, BSA-enriched, amino acid free medium supported a high level of development of cell zygotes as compared to control samples/media.

TABLE 4
Development of 1 cell zygotes in the new buffering
system, compared with 25 mM HCO₃⁻

| | No Zygotes | No 2 cells | No Blasts | % Blasts/2 cell |
|---|---|---|---|---|
| Control media | 20 | 16 | 11 | 69 |
| New buffer | 20 | 17 | 16 | 94 |

### Example 3

### Determination of Stability of pH when Media is Removed from a 6% CO₂ Incubator

K-CKBM medium was modified to include a range of HEPES/MOPS concentrations as shown in Table 5.

TABLE 5

| | | | | | | | Finished |
|---|---|---|---|---|---|---|---|
| | | | | | | Batch | Product pH |
| Specifications | HEPES | MOPS | NaHCO3 | NaCl | | Number | and Osmo |
| In process | 3.75 mM | 4 mM | 25 mM | 103 | mM | TR0502 | pH 7.211 and |
| pH 7.2-7.6 | HEPES Na Salt, | MOPS Na salt, | NaHCO3 | | | | Osmo 289 |
| Osmo 285-295 | 3.75 mM | 3.5 mM | | | | | |
| | HEPES acid | MOPS acids | | | | | |
| | 7.5 mM | 8 mM | 25 mM | 92.5 | mM | TR0503 | pH 7.238 and |
| | HEPES Na salt, | MOPS Na salt, | NaHCO3 | | | | Osmo 292 |
| | 7.5 mM | 7 mM | | | | | |
| | HEPES acid | MOPS acids | | | | | |
| | 12.5 mM | 13.33 mM | 25 mM | 77 | mM | TR0507 | pH 7.247 and |
| | HEPES Na salt, | MOPS Na salt, | NaHCO3 | | | | Osmo 291 |
| | 12.5 mM | 11.67 mM | | | | | |
| | HEPES acid | MOPS acids | | | | | |
| | 0 | 0 | 25 | 110 | mM | TR0506 | pH 7.306 and |
| | | | | | | | Osmo 289 |

20 ml samples of the above four buffers having varying HEPES/MOPS concentrations were placed in pH buffer cups and incubated overnight at 37° C. in a 6% CO₂ incubator. Following the overnight incubation, the initial pH of the samples was measured in the incubator. Two samples were measured in parallel using two pH meters.

Once the pH was measured, the samples were removed from the 6% CO₂ incubator and placed into a non-CO₂ incubator or keep at room temperature. The monitoring of the pH was initiated immediately following the transfer of the samples from the CO₂ incubator into a non-CO₂ incubator (at 37° C.) or room temperature. The pH of the samples was measured again to determine stability of pH when environment conditions change.

The pH values measured at room temperature are provided in Table 6 below.

TABLE 6
pH values at Room Temperature

| Min- | Control 0 mM | 15 mM | 25 mM | 50 mM |
|---|---|---|---|---|
| utes | HEPES/MOPS | HEPES/MOPS | HEPES/MOPS | HEPES/MOPS |
| 0 | 7.419 | 7.359 | 7.356 | 7.302 |
| 1 | 7.415 | 7.356 | 7.36 | 7.306 |
| 2 | 7.415 | 7.363 | 7.367 | 7.309 |
| 3 | 7.416 | 7.368 | 7.371 | 7.314 |
| 4 | 7.417 | 7.373 | 7.373 | 7.317 |
| 5 | 7.42 | 7.378 | 7.372 | 7.323 |
| 10 | 7.439 | 7.4 | 7.385 | 7.347 |
| 15 | 7.453 | 7.423 | 7.4 | 7.364 |
| 20 | 7.486 | 7.448 | 7.409 | 7.373 |
| 30 | 7.54 | 7.476 | 7.444 | 7.397 |
| 45 | 7.641 | 7.522 | 7.449 | 7.427 |
| 60 | 7.657 | 7.53 | 7.481 | 7.452 |
| 75 | 7.706 | 7.559 | 7.495 | 7.478 |
| 90 | 7.742 | 7.599 | 7.508 | 7.486 |
| 105 | 7.781 | 7.648 | 7.518 | 7.501 |
| 120 | 7.82 | 7.655 | 7.53 | 7.52 |
| 150 | 7.846 | 7.673 | 7.547 | 7.533 |
| 180 | 7.902 | 7.693 | 7.554 | 7.553 |
| 210 | 7.947 | 7.707 | | 7.562 |

The pH values measured at 37° C. in the non-CO₂ incubator are provided in Table 7 below.

TABLE 7
pH values at 37° C.

| | Control 0 mM | 15 mM | 25 mM | 50 mM |
|---|---|---|---|---|
| | HEPES/MOPS | HEPES/MOPS | HEPES/MOPS | HEPES/MOPS |
| 0 | 7.42 | 7.34 | 7.248 | 7.341 |
| 1 | 7.413 | 7.335 | 7.253 | 7.335 |
| 2 | 7.415 | 7.338 | 7.255 | 7.337 |
| 3 | 7.418 | 7.347 | 7.259 | 7.337 |
| 4 | 7.425 | 7.358 | 7.26 | 7.337 |
| 5 | 7.434 | 7.362 | 7.261 | 7.338 |
| 10 | 7.512 | 7.406 | 7.268 | 7.343 |
| 15 | 7.576 | 7.415 | 7.278 | 7.35 |
| 20 | 7.616 | 7.405 | 7.286 | 7.353 |
| 30 | 7.674 | 7.458 | 7.299 | 7.362 |
| 45 | 7.678 | 7.506 | 7.321 | 7.376 |
| 60 | 7.722 | 7.527 | 7.338 | 7.399 |
| 75 | 7.768 | 7.558 | 7.352 | 7.41 |
| 90 | 7.824 | 7.591 | 7.366 | 7.422 |
| 105 | 7.881 | 7.631 | 7.376 | 7.448 |
| 120 | 7.926 | 7.644 | 7.392 | 7.455 |
| 150 | 7.977 | 7.661 | 7.416 | 7.481 |
| 180 | 8.074 | 7.713 | 7.439 | 7.502 |
| 210 | 8.159 | 7.753 | 7.459 | 7.526 |

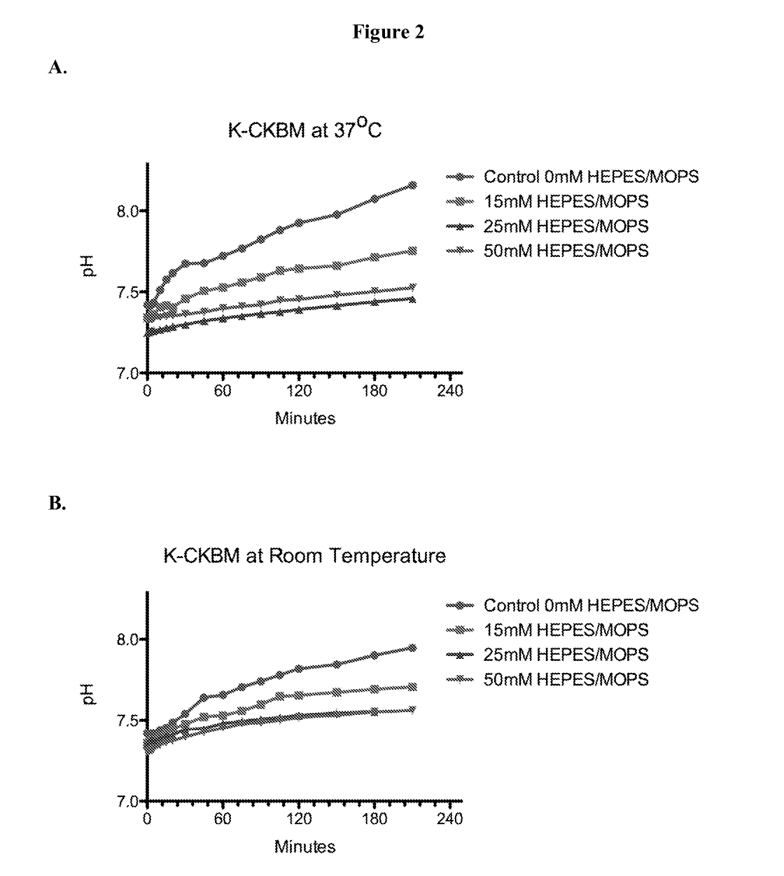

As shown in FIG. 2A, the pH of the samples containing 25 mM and 50 mM of HEPES/MOPS did not change once the samples were transferred from the 37° C./6% CO₂ incubator to a non-CO₂ incubator also at 37° C. The pH of the samples containing 15 mM HEPES/MOPS increased slowly overtime (a few tenths of a digit within 210 minutes of the testing period). The pH of the control samples rose immediately following the change of the incubators and was significantly higher than the test samples at the 210 minute mark.

Similar results were observed when the samples were removed from the 37° C./6% CO₂ incubator and kept at room temperature; see FIG. 2B. Specifically, the pH of the samples containing 25 mM and 50 mM of HEPES/MOPS did not change significantly once the samples were removed from the 37° C./6% CO₂ incubator and kept at room temperature. The pH of the samples containing 15 mM HEPES/MOPS increased slowly overtime (a few tenths of a digit within the 210 minutes of testing). The pH of the control sample rose immediately following the change of the environment and was significantly higher than the test samples at the 210 minute mark.

1. A method for continuous stabilization of pH of cell culture medium notwithstanding change from the first environment conditions to the second environment conditions, comprising providing a buffering system, the buffering system comprising sodium bicarbonate at a concentration from 4 to 30 mM, and at least two zwitterionic buffers at a concentration from 1 to 50 mM,
wherein the pH is 7.0-7.6, and
wherein the first and the second environment conditions are distinct.

2. The method of claim 1, wherein the zwitterionic buffers are selected from the group consisting of 3-N-morpholinopropane sulfonic acid (MOPS); N-2-Hydroxyethylpiperazine-N′-2-ethanesulfonic acid (HEPES); and N,N-bis2-Hydroxyethyl-2-aminoethanesulfonic acid (BES); 1,3-bistris(Hydroxymethyl)methylaminopropane (BIS-TRIS PROPANE); 2-bis(2-Hydroxyethyl)aminoethanesulfonic acid); (N-trisHydroxymethylmethyl-2-aminoethane-sulfonic acid (TES); 2-(2-Hydroxy-1,1-bis(hydroxymethyl)ethylamino)ethanesulfonic acid; 3-N,N-bis(2-Hydroxyethyl)methylamino-2-hydroxy-propanesulfonic acid) (DIPSO); 3-N-tris(Hydroxymethyl)methylamino-2-hydroxypropanesulfonic acid) (TAPSO); trisHydroxymethylaminomethane),(2-amino-2-hydroxymethyl)-1,3-propanediol) (TRIZMA); N-2-Hydroxyethylpiperazine-N′-2-hydroxy-propanesulfonic acid (HEPPSO); Piperazine-N,N′-bis2-hydroxypropanesulfonic acid (POPSO); N2-Hydroxyethylpiperazine-N′-3-propane-sulfonic acid (HEPPS); and Triethanolamine, (2,2′2″-Nitrilotriethanol) (TEA).

3. The method of claim 1, wherein the first environment conditions are CO₂-enriched environment conditions and the second environment conditions are ambient environment conditions.

4. The method of claim 1, wherein the first environment conditions are CO₂-enriched environment conditions and the second environment conditions are CO₂-reduced environment conditions.

5. The method of claim 1, wherein the first environment conditions are ambient environment conditions and the second environment conditions are CO₂-enriched environment conditions.

6. The method of claim 1, wherein the first environment conditions are ambient environment conditions and the second environment conditions are CO₂-reduced environment conditions.

7. The method of claim 1, wherein the first environment conditions are CO₂-reduced environment conditions and the second environment conditions are ambient environment conditions.

8. The method of claim 1, wherein the first environment conditions are CO₂-reduced environment conditions and the second environment conditions are CO₂-enriched environment conditions.

9. A cell culture buffering system for continuous stabilization of pH of cell culture medium from first environment conditions to second environment conditions comprising:
4 to 30 mM of sodium bicarbonate, and
at least two zwitterionic buffers at a concentration from 1 to 50 mM,
wherein the pH is 7.0-7.6, and
wherein the first and the second environment conditions are distinct.

10. The cell culture buffering system of claim 9, comprising 7.5 nM 3-N-morpholinopropane sulfonic acid (MOPS), 7.5 nM 3-N-morpholinopropane sulfonic acid (MOPS), and 25 nM NaHCO₃.

11. The cell culture buffering system of claim 9, wherein the zwitterionic buffers are selected from the group consisting of 3-N-morpholinopropane sulfonic acid (MOPS); N-2-Hydroxyethylpiperazine-N′-2-ethanesulfonic acid (HEPES); and N,N-bis2-Hydroxyethyl-2-aminoethanesulfonic acid (BES); 1,3-bistris(Hydroxymethyl)methylaminopropane (BIS-TRIS PROPANE); 2-bis(2-Hydroxyethyl)aminoethanesulfonic acid); (N-trisHydroxymethylmethyl-2-aminoethane-sulfonic acid (TES); 2-(2-Hydroxy-1,1-bis(hydroxymethyl)ethylamino)ethanesulfonic acid; 3-N,N-bis(2-Hydroxyethyl)methylamino-2-hydroxy-propanesulfonic acid) (DIPSO); 3-N-tris(Hydroxymethyl)methylamino-2-hydroxypropanesulfonic acid) (TAPSO); trisHydroxymethylaminomethane),(2-amino-2-(hydroxymethyl)-1,3-propanediol) (TRIZMA); N-2-Hydroxyethylpiperazine-N′-2-hydroxy-propanesulfonic acid (HEPPSO); Piperazine-N,N′-bis2-hydroxypropanesulfonic acid (POPSO); N2-Hydroxyethylpiperazine-N′-3-propane-sulfonic acid (HEPPS); and Triethanolamine, (2,2′2″-Nitrilotriethanol) (TEA).

12. The cell culture buffering system of claim 9, wherein the buffering system is suitable for use to support the development of cells.

13. The cell culture buffering system of claim 9, wherein the buffering system is suitable for use to support the development of embryos.

14. The cell culture buffering system of claim 9, wherein the buffering system is suitable for use to support the development of zygotes.

15. The cell culture buffering system of claim 9, wherein the buffering system is for use as a cryopreservation buffer.

16. The cell culture buffering system of claim 9, wherein the buffering system is for use as an in-vitro maturation buffer.

17. The cell culture buffering system of claim 9, wherein the buffering system is for use with follicular cultures.

18. The cell culture buffering system of claim 9, wherein the buffering system is for use in assisted reproduction procedures.

19. The cell culture buffering system of claim 9, wherein the buffering system is for use with granulosa cells, theca cells, cumulus cells, germinal vesicle oocytes, methaphase I oocytes, and metaphase II oocytes and all stages of preimplantation embryo development and stem cells derived from any of embryonic cells or cells either collected from tissue or induced from any non-stem cell, and sperm cells.

20. A method for continuous stabilization of pH of cell culture medium comprising ejaculated sperm cells, notwithstanding change from the first environment conditions to the second environment conditions, comprising providing a buffering system, the buffering system comprising sodium bicarbonate at a concentration from 4 to 30 mM, and at least two zwitterionic buffers at a concentration from 1 to 50 mM,
wherein the pH is 7.0-7.6, and
wherein the first and the second environment conditions are distinct.

21. The method of claim 20, wherein the sperm cells are in a non-cryopreserved state.

22. The method of claim 20, wherein the buffering system provides for a continuous stabilization of pH of the cell culture medium over a time period of about 24 to about 48 hours without a temperature control.

23. A method for transporting ejaculated sperm cells in a cell culture medium from a first environment to a second environment without a temperature control and notwithstanding change from the first environment conditions to the second environment conditions, comprising
providing a buffering system, the buffering system comprising sodium bicarbonate at a concentration from 4 to 30 mM, and at least two zwitterionic buffers at a concentration from 1 to 50 mM,
wherein the cell medium has a pH of 7.0-7.6, and
wherein the first and the second environment conditions are distinct.
